(19) United States
(12) Patent Application Publication
(10) Pub. No.: US 2010/0075320 A1
(43) Pub. Date: Mar. 25, 2010

(54) PAXILLIN MUTATIONS, METHODS OF ASSESSING RISK OF METASTASIS AND METHODS OF STAGING TUMORS

(76) Inventor: Ravi Salgia, Park Ridge, IL (US)

Correspondence Address: MICHAEL BEST & FRIEDRICH LLP, ONE SOUTH PINCKNEY STREET, P O BOX 1806, MADISON, WI, 53701, US

(21) Appl. No.: 12/526,870

(22) PCT Filed: Feb. 15, 2008
(86) PCT No.: PCT/US08/54114

§ 371 Date: Sep. 2, 2009

Related U.S. Application Data
(60) Provisional application No. 60/890,120, filed on Feb. 15, 2007.

(51) Int. Cl. C12Q 1/68; C07H 21/04

(52) U.S. Cl. 435/6; 536/23.1

(57) ABSTRACT

Disclosed herein are methods for assessing risk of metastasis of a tumor in a mammal by determining the paxillin gene copy number per cell in the tumor or by detecting the presence of a paxillin mutation in the tumor. The presence of an increased paxillin gene copy number or a paxillin mutation is indicative of an increased risk of metastasis. Methods of staging tumors and methods of reducing invasiveness or metastasis of a cancer cell are also provided. Oligonucleotides comprising a paxillin mutation and antibodies capable of recognizing a paxillin mutant are disclosed.

### CROSS-REFERENCE TO RELATED APPLICATIONS

This application claims priority to U.S. Provisional Application No. 60/890,120, filed Feb. 15, 2007, which is incorporated herein by reference in its entirety.

### STATEMENT REGARDING FEDERALLY SPONSORED RESEARCH

This invention was made with government support under R01 grant numbers, CA100750-04 and CA125541-01, awarded by the NIH/National Cancer Institute. The government has certain rights in the invention.

### BACKGROUND

The cytoskeleton plays an important role in abnormal growth, invasion, and metastasis of malignant tumors. Cellular adhesions between tumor cells and normal cells, and between adjacent tumor cells, are essential for the progression of cancer. Paxillin is involved in forming cellular adhesions by forming a structural link between the extracellular matrix and the actin cytoskeleton. As a central protein within the focal adhesion, paxillin, in normal cells, acts as a scaffold protein that provides multiple docking sites at the plasma membrane for an array of signaling and structural proteins. Paxillin provides a platform for protein tyrosine kinases, including focal adhesion kinase (FAK) and Src, which are activated as a result of adhesion or growth factor stimulation.

Gene disruption of paxillin in mice affects the development of both extra-embryonic and embryonic structures. Paxillin null cells have abnormal focal adhesions, an altered cortical cytoskeleton, decreased tyrosine phosphorylation of FAK and p130Cas, decreased efficiency of FAK localization to focal adhesions, decreased activation of MAPK, a decreased rate of spreading, and decreased cell migration. In addition to interacting with cytoskeletal proteins, paxillin interacts with several oncoproteins such as E6, v-Src, and BCR-ABL.

### SUMMARY OF THE INVENTION

In one aspect, methods for assessing risk of metastasis of a tumor in a mammal are provided. The methods include determining the paxillin gene copy number in cells of the tumor. The presence of an increased number of paxillin gene copies in the tumor cells is indicative of an increased risk of metastasis.

In another aspect, methods are provided for assessing risk of metastasis of a tumor in a mammal by detecting the presence of a paxillin mutation in cells of the tumor. The presence of a paxillin mutation may be indicative of an increased risk of metastasis.

In yet another aspect, methods are provided for staging a tumor in a mammal. The methods include detecting the presence of a paxillin mutation in the tumor and then correlating the presence of the paxillin mutation in the tumor with the stage of the tumor.

In a further aspect, isolated oligonucleotides at least 16 bases in length are provided. The oligonucleotides comprise a paxillin mutation at a nucleotide position of SEQ ID NO:36 selected from the group consisting of 162, 169, 191, 210, 211, 213, 214, 223, 224, 229, 388, 453, 489, 772, 788, 838, 902, 963, 1137, 1140, 1147, 1220, 1236, 1256, 1257, 1269, 1297, 1341, 1528, 1534, 1591, and 1601.

In a still further aspect, methods are provided for reducing invasiveness or metastasis of a cancer cell. These methods include contacting the cancer cell with a therapeutically effective amount of a paxillin-specific RNAi-inducing agent. An RNAi-inducing agent recognizes a paxillin mutation and affects paxillin transcription or translation.

In yet another aspect, antibodies capable of binding a paxillin epitope comprising a mutation are provided.

### BRIEF DESCRIPTION OF THE DRAWINGS

FIG. 1A is a bar graph depicting the frequency and relative level of paxillin expression in normal lung and primary/metastatic NSCLC. Star indicates P<0.01.

FIG. 1B is a bar graph depicting the frequency and relative level of paxillin expression in NSCLC subtypes such as large cell carcinoma, squamous cell carcinoma and adenocarcinoma. Normal adjacent lungs were negative for paxillin expression (P=0.025, Chi-Square test).

FIG. 1C is a bar graph depicting the frequency and relative level of paxillin expression in samples from different stages of NSCLC.

FIG. 1D is a bar graph depicting the frequency and relative level of paxillin expression in normal head and neck, head and neck dysplasia and head and neck tumors.

FIG. 1E is a bar graph depicting the frequency and relative level of paxillin expression in mesothelioma tumor tissues.

FIG. 2A shows photographs of immunoblots for protein expression from lysates of the NSCLC and bronchial epithelial (NHBE, BEAS-2B) cell lines using antibodies against paxillin, c-Met, EGFR, and β-actin (loading control).

FIG. 2B shows photographs of immunoblots for protein expression from lysates of mesothelioma and control (MeT-5A) cell lines using antibodies against paxillin, c-Met, and β-actin (loading control).

FIG. 2C is a table showing the results of fluorescent in situ hybridization analysis (FISH) on the indicated NSCLC cell lines showing positive increased copy numbers (>4), clustered amplification (>10) or normal (diploid) PXN, MET and EGFR genes.

FIG. 2D is a bar graph showing the distribution of PXN, MET and EGFR gene copy numbers (>4) for each non small cell lung carcinoma subtype as assessed by real time PCR analysis. (NB: PXN, MET and EGFR are the genes encoding paxillin, c-Met tyrosine kinase receptor and the epidermal growth factor receptor, respectively). Change in PXN copy numbers was highly correlated with MET copy numbers (Spearman r=0.40, P=0.001).

FIG. 3A is a schematic diagram of paxillin showing the somatic missense mutations identified in cancer tissues and cancer cell lines in the context of the functional domains of paxillin. Arrowheads indicate the location of missense mutations. Positions of amino acid and nucleotide changes in the paxillin mutant samples are listed in Table 2.

FIG. 3B is a bar graph showing the frequency of paxillin mutations among NSCLC samples from different ethnic groups (P=0.017, Chi-Square test).

FIG. 3C is a bar graph showing the number of each class of mutations found in human cancer tissue specimens and cancer cell lines in the paxillin gene.

FIG. 4A is a graph depicting cell growth as a percentage of control in H522 cells transfected with EGFP vector, EGFP fused to wild type paxillin, and EGFP fused to A127T paxillin shown for the indicated starvation time. *P<0.001. The inset photograph depicts an immunoblot with paxillin antibody (5H11) demonstrating significant expression of EGFP fused to paxillin (95 kDa).

FIG. 4B is a bar graph depicting anchorage independent cell growth and colony formation of transfected H522 cells with vector, wild-type paxillin, and A127T mutant paxillin. The inset photograph depicts a representative colony formation assay of three independent experiments. The results are shown as the number of colonies obtained in control, wild-type paxillin and A127T paxillin. Error bar denotes S.E.M. ^{#}P=0.0005; *P<0.0001.

FIG. 4C is a bar graph showing cell viability of SK-LU-1 and BEAS-2B cells transfected with control siRNA (scrambled sequences) or with paxillin specific siRNA duplexes (100 pmol/mL). Columns, mean percentage of cell growth; bars, S.E.M (n=3). The inset photograph is an immunoblot analysis to confirm siRNA-directed knockdown of paxillin expression in BEAS-2B and SK-LU-1 cells.

FIG. 5A is a set of confocal images of H522 cells transfected with EGFP vector (control), EGFP fused to wild type paxillin and EGFP fused to A127T paxillin. Nuclear location is indicated with letter “N”. Boxed regions show 2× magnification.

FIG. 5B is a set of confocal images showing cells that were stained for F-actin and paxillin and projections of optical sections corresponding to the plane of adhesion are shown. Boxed regions are shown magnified 3 times and contrast enhanced. Bar, 20 μm.

FIG. 6A is a graph depicting the tumorigenicity of H522-control vector (diamond), H522-wt paxillin (square) and H522-A127T paxillin (triangle) transfected H522 cells in nude mice. Each point represents the mean value for six tumors per group. *P=0.0016 (as compared to control vector). Data analyzed by a two-tailed Student's t-test.

FIG. 6B is a set of bar graphs showing a semi-quantitative analysis of tumor tissues stained for Ki67, trichrome and CD31. Error bar denotes SEM.

### DETAILED DESCRIPTION

As described in the Examples, paxillin expression was found to be increased in subpopulations of a variety of tumors and cancer cell lines. The results show that increased expression of paxillin is correlated with increased tumor invasiveness and metastasis. It was also found that paxillin is overexpressed in higher grade tumors, relative to lower grade tumors and normal cells. Thus, paxillin expression may be used to assess tumor invasiveness, risk of metastasis, or to stage tumors. As one of skill in the art will appreciate, any suitable method for evaluating paxillin expression may be used to evaluate paxillin expression according to the methods of the invention including, but not limited to, real time RT-PCR, Northern analysis and Western analysis.

The Examples also demonstrate amplification of the paxillin gene in many cancer cell lines. Paxillin gene amplification may be indicative of increased tumor invasiveness, risk of metastasis or tumor stage. Paxillin gene copy number may be assessed by any means known to those of skill in the art, including but not limited to, FISH and real time PCR. In the

Examples, both FISH and real time PCR were used to demonstrate paxillin gene amplification. In particular gene amplification was noted in non-small cell lung cancer (NSCLC) derived cells, such as large cell carcinomas.

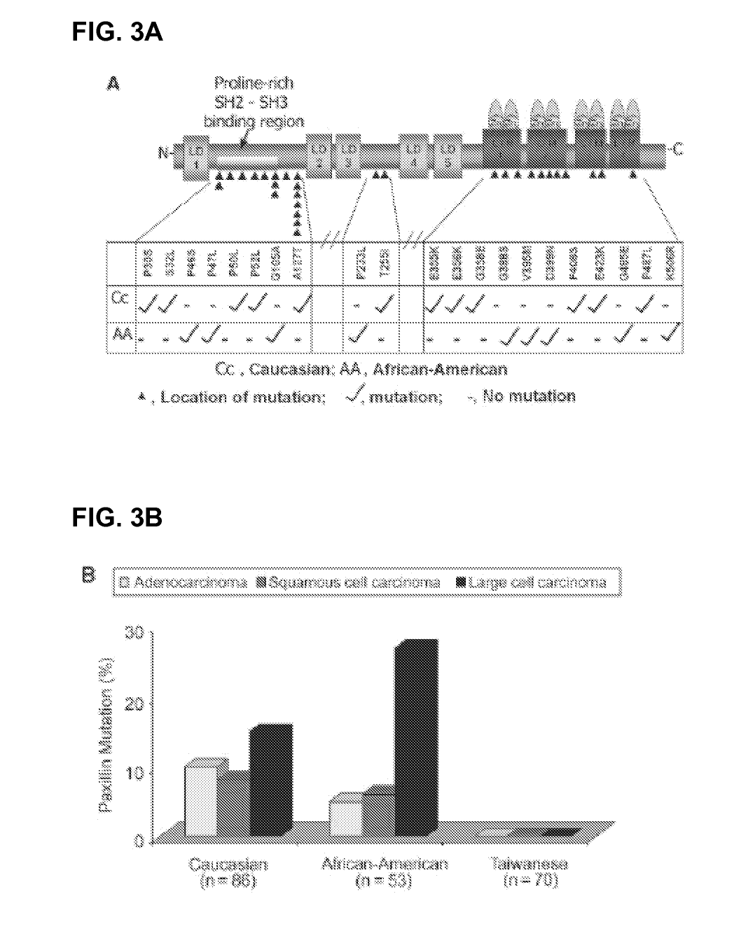

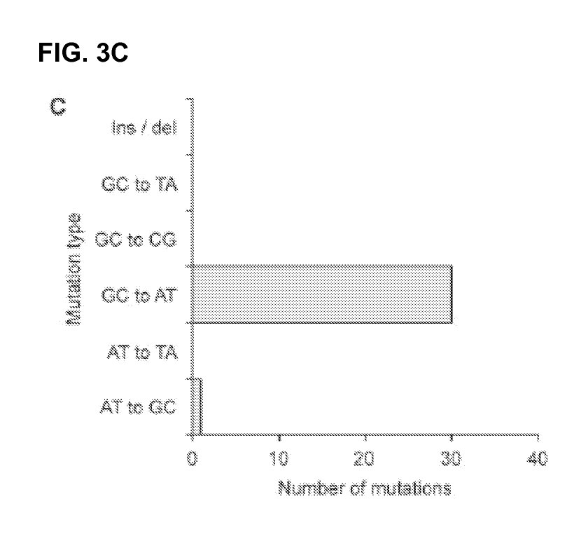

Additionally, several distinct populations of tumors and cell lines were screened for paxillin mutants. Paxillin mutants were identified in many types of cancers, in either the primary tumor or in tumor-derived cancer cell lines. Mutants occurred primarily between the LD motifs or in the LIM domains, as shown in FIG. 3A. Mutations were particularly common between the LD1 and LD2 motifs and between the LD3 and LD4 motifs.

Mutations in the paxillin gene may result in an amino acid change. These mutations include, but are not limited to, P30S, S32L, P46S, P46L, P47L, P47S, P50L, P52L, G105D, A127T, G139S, P233L, T255I, G297R, E355K, E356K, G358E, G388S, V395M, D399N, F4085, E423K, P487L, and K506R. The mutations are noted in reference to the amino acid sequence of paxillin in SEQ ID NO: 35.

Mutations in the paxillin gene may be single nucleotide polymorphisms (SNPs) that do not result in an amino acid change in the protein. These mutations include, but are not limited to, change at a nucleotide of SEQ ID NO: 36 selected from the group consisting of nucleotide position numbers 191, 224, 788, 902, 1220, 1256 and 1601. The mutations are noted in reference to the nucleotide sequence of paxillin in SEQ ID NO: 36. As indicted in FIG. 3C, the most common type of mutation in paxillin found in cancer cell lines and tissues was a GC to AT transition.

Mutations may be detected by any suitable means, including but not limited to, by sequencing the full length paxillin coding sequence or a portion thereof, and by performing a restriction fragment length polymorphism analysis. It is also envisioned that specific paxillin mutations could be detected by determining whether antibodies raised against an epitope comprising a mutation bind to a tissue sample. In addition, oligonucleotides capable of specifically binding the mutated paxillin gene may be used as a probe under high stringency conditions to differentiate the wild-type and mutant paxillin gene. High stringency conditions would include any conditions in which a single nucleotide change can be detected. Alternatively, paxillin RNA or DNA from target cells may be amplified by PCR, labeled and the resulting polynucleotides used to probe an oligonucleotide array containing oligonucleotides comprising paxillin mutations. The presence of paxillin comprising mutations in a tumor cell is indicative of increased tumor invasiveness, increased risk of metastasis or may be used to determine the stage of tumors.

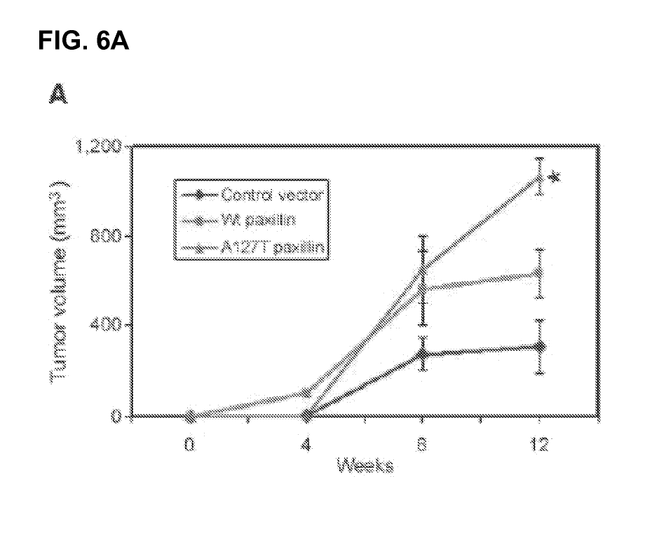

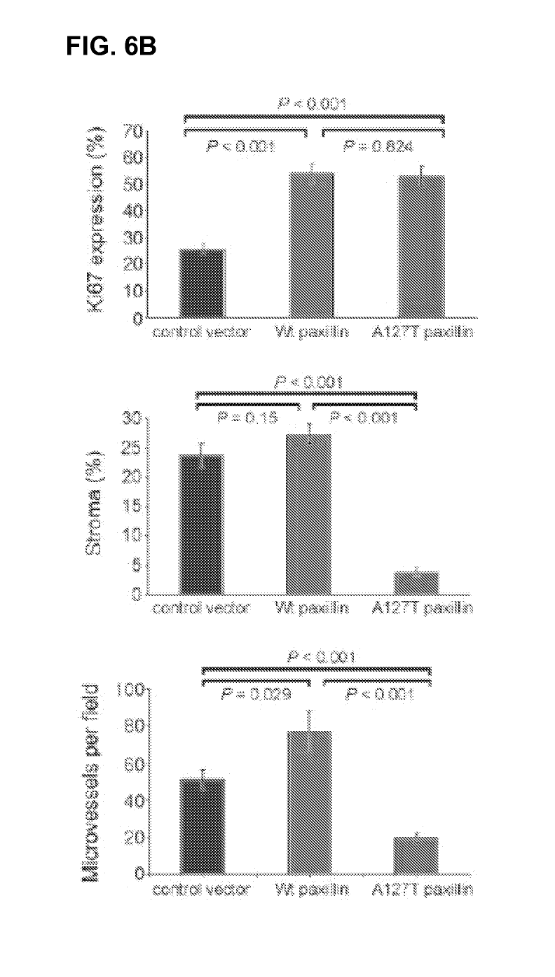

It was discovered that the paxillin mutation that occurred with the greatest frequency among the tested tumors and cell lines is A127T paxillin. In an in vivo mouse model, expression of this A127T paxillin is associated with increased angiogenesis, increased tumor invasiveness and increased tumor growth as compared to cells expressing similar levels of wild-type paxillin. See FIG. 6.

It is reasonably expected that overexpression of paxillin due to gene amplification or paxillin mutations play roles in numerous types of cancers including, but not limited to lung cancer, prostate cancer, testicular cancer, brain cancer, skin cancer, colon cancer, rectal cancer, gastric cancer, esophageal cancer, tracheal cancer, head and neck cancer, pancreatic cancer, liver cancer, breast cancer, ovarian cancer, lymphoid cancer, leukemia, cervical cancer, vulvar cancer, melanoma, mesothelioma, renal cancer, bladder cancer, thyroid cancer, bone cancers or soft tissue cancers. Thus, the present invention is believed to be generally applicable to any type of cancer in which overexpression of paxillin due to gene copy number amplification or paxillin mutation occurs. Suitably, the lung cancer is a small cell lung cancer or a NSCLC. Suitably a NSCLC is a large cell carcinoma, an adenocarcinoma or a squamous cell carcinoma.

In another aspect, the present invention comprises an isolated oligonucleotide of at least 16 bases in length which includes a mutant paxillin sequence. The oligonucleotide may be useful as a probe, primer or RNAi. The oligonucleotide may be labeled by any means known to those of skill in the art including, but not limited to a fluorescent or radioactive label. The mutant paxillin sequence may encompass any of the paxillin mutants disclosed herein, including but not limited to mutations at a nucleotide position of SEQ ID NO:36 selected from the group consisting of 162, 169, 191, 210, 211, 213, 214, 223, 224, 229, 388, 453, 489, 772, 788, 838, 902, 963, 1137, 1140, 1147, 1220, 1236, 1256, 1257, 1269, 1297, 1341, 1528, 1534, 1591, and 1601. The present invention also encompasses antibodies specific for a paxillin epitope comprising a point mutation as described above or in the Examples.

Thus, paxillin is a promising target in the treatment of cancers. It is envisioned that cancers in which paxillin is overexpressed may be treated by contacting or delivering to the tumor cells an agent that inhibits paxillin expression. For example, a paxillin-specific RNAi-inducing agent may be used to inhibit paxillin expression. Suitably the RNAi inducing agent recognizes a paxillin mutant. Treatment with a therapeutically effective amount of the RNAi-inducing agent may reduce invasiveness of the tumor, reduce metastasis of the tumor or limit the growth of the tumor.

An “RNAi-inducing agent,” as the term is used herein and in the art, encompasses RNA molecules or vectors capable of inducing expression of RNA molecules. The presence of RNAi-inducing agents in a cell results in RNA interference and leads to reduced expression of a transcript to which the RNAi-inducing agent is targeted. Suitably, for some embodiments, delivery of an RNAi-inducing agent reduces expression of the target transcript to a level that confers a therapeutic effect. Delivery of an RNAi-inducing agent suitably reduces expression of the target transcript by at least about 10%, at least about 20%, at least about 30%, at least about 40%, at least about 50%, at least about 60%, at least about 70%, at least about 80%, at least about 90%, at least about 95%, at least about 98%, at least about 99% or 100%, relative to expression of the transcript in an appropriate control cell. RNAi-inducing agents specifically include short interfering RNA (“siRNA”), short hairpin RNA (“shRNA”), and RNAi-inducing vectors, each of which is defined below. Selection of appropriate target sequences for RNAi may take into account factors such as synthetic considerations, avoiding interference with transcripts other than the target transcript, and other considerations, as described by Manoharan, (Current Opinion in Chemical Biology 2004, 8:570-579 (2004)), which is incorporated herein by reference in its entirety.

A “short, interfering RNA,” or “siRNA,” comprises an RNA duplex that is about 19 to about 27 base pairs in length and optionally further comprises one or two single-stranded overhangs. As is appreciated, siRNA can be chemically synthesized, or can be transcribed in vitro from a DNA template, or in vivo from an RNA precursor. An siRNA may be formed from two RNA molecules that hybridize, or may alternatively be generated from a single RNA molecule that includes a self-hybridizing portion. It is generally preferred that free 5′ ends of siRNA molecules have phosphate groups, and free 3′ ends have hydroxyl groups. The duplex portion of an siRNA may, but typically does not, contain one or more unpaired nucleotides. One strand of an siRNA includes a portion that hybridizes with a target transcript. In certain embodiments of the invention, one strand of the siRNA is precisely complementary with a region of the target transcript, meaning that the siRNA hybridizes to the target transcript without a single mismatch. In other embodiments, one or more mismatches between the siRNA and the targeted portion of the target transcript may exist. In most embodiments of the invention in which perfect complementarity is not achieved, it is generally preferred that any mismatches be located at or near the siRNA termini. An siRNA used in accordance with the invention is suitably hybridizable to a target transcript and capable of inducing its degradation.

The term “short hairpin RNA” refers to an RNA molecule comprising at least two complementary portions hybridized or capable of hybridizing to form a double-stranded (duplex) structure of sufficient length to mediate RNAi (typically about 19-27 base pairs in length), and at least one single-stranded portion, typically between approximately 1 and 10 nucleotides in length, that forms a loop structure. The duplex portion may, but typically does not, contain one or more unpaired nucleotides. Not to be bound by theory, it is thought that shRNAs are precursors of siRNAs and are, in general, similarly capable of inhibiting expression of a target transcript.

As used herein, “an RNAi-inducing vector” is a vector the presence of which in a cell results in transcription of one or more RNAs that self-hybridize or hybridize to each other to form an shRNA or siRNA. In general, the term generally encompasses any construct comprising a polynucleotide operably linked to expression signal(s) so that one or more RNA molecules that hybridize or self-hybridize to form an siRNA or shRNA are transcribed when the vector is present within a cell. Thus, the vector provides a template for intracellular synthesis of the RNAi-inducing agent or precursors thereof. An RNAi-inducing vector is considered to be targeted to a transcript if presence of the vector within a cell results in production of one or more RNAs that hybridize to each other or self-hybridize to form an siRNA or shRNA that is targeted to the transcript, i.e., if presence of the vector within a cell results in production of one or more siRNAs or shRNAs targeted to the transcript. Genetic constructs for the delivery of siRNA molecules are described, for example, in U.S. Pat. No. 6,573,099, which is incorporated herein by reference. A further example of the use of shRNA expression plasmids to reduce gene expression in vivo in rats has been described by Zhang et al. (J. Gene Med. 5:1039-1045, 2003), which is also incorporated herein by reference.

In particular embodiments, the RNAi-inducing agent is suitably stabilized by chemical modification. For example, siRNAs may be crosslinked to increase half-life in the body. For example, a 3′ OH terminus of one of the strands of double-stranded siRNA can be modified, or the two strands can be crosslinked and modified at the 3′ OH terminus. The siRNA derivative can contain a single crosslink or multiple crosslinks. Additionally, stability may be enhanced by including nucleotide analogs at one or more free ends in order to reduce digestion, e.g., by exonucleases. The inclusion of deoxynucleotides, e.g., pyrimidines such as deoxythymidines at one or more free ends, may serve this purpose. It will further be appreciated by those of ordinary skill in the art that effective siRNA agents for use in accordance with certain embodiments of the invention may comprise one or more moieties that are not nucleotides or nucleotide analogs. Further suitable chemical modifications are described by, e.g., Manoharan, Current Opinion in Chemical Biology 2004, 8:570-579 (2004), incorporated herein by reference in its entirety.

In a further embodiment, micro RNA (“miRNA”) is delivered to cells according to the invention. As used herein, “micro RNA” or “miRNA” is an RNAi-inducing agent that refers to single-stranded, non-coding RNA molecules of about 19 to about 27 base pairs that regulate gene expression in a sequence specific manner.

In another embodiment of the invention, an alternative RNAi-inducing agent is an antagomir which is delivered to cells according to the invention. As used herein, an “antagomir” is an RNA species that is complementary to a target endogenous miRNA. Suitably, upon binding of an antagomir to its complement miRNA, the action of the miRNA is antagonized.

The RNAi-inducing agents suitably recognize a paxillin mutant and are capable of decreasing paxillin expression in cells. Decreased paxillin expression in cancer cells may result in decrease growth of the cancer cells, decreased invasiveness of the cancer cells or the tumor, decreased metastasis and decreased angiogenesis. Paxillin mutants that may be recognized by the RNAi include, but are not limited to, the mutations defined above and in the Examples which follow.

The following Examples are meant to be illustrative and do not limit the scope of the invention as defined in the claims.

### Examples

### Materials and Methods

### Cell Lines and Tissue Specimens

H441, SK-LU-1, H1993, A549, H522, H358, H661, SW1573, H2170, SW-900, NHBE and BEAS-2B cell lines were obtained from the American Type Culture Collection (ATCC, Rockville, Md.) and cultured as described by the ATCC. Genomic DNA (total 483) was isolated from 191 lung cancer tissues (from Caucasians and African-Americans), 70 lung cancer tissues from Asians, 151 non-lung tumor tissues, and 71 lung cancer cell lines by standard procedures (Ma P C, Jagadeeswaran R, Jagadeesh S, et al. Cancer Res 2005;65:1479-88).

### DNA Sequencing and Mutational Analysis

11 pairs of PCR primers to cover the 11 exons spanning the entire coding region of paxillin were used (See Table 1) and the paxillin coding region from cancer DNA specimens and cancer cell lines was sequenced (Ma P C, Kijima T, Maulik G, et al. Cancer Res 2003;63:6272-81.). The adjacent “normal” tissues of the surgically resected tumor specimens were identified histopathologically and sequenced as well. The numbering of the nucleotide positions was relative to the first base of the translational initiation codon according to the full-length human alpha paxillin cDNA (See SEQ ID NO: 36). All mutations were confirmed by sequencing in both directions.

TABLE 1
Primers for mutational analysis and quanti-
tative real-time PCR analysis.
Primers for PXN mutational analysis

| Exon | Forward | Reverse |
|---|---|---|
| 1 | 5′ CAGCTAGCGCGACCCTGAG | 5′ GAATGGTCCTGCACCAGTA |
| | C 3′ | ATATG 3′ |
| 2 | 5′GGTGCTAGAAGGCATTAACT | 5′ CTTAGCTGGCAGGGGCTTA |
| | CTTG 3′ | G 3′ |
| 3 | 5′ GTGGAAATTCCCTGGTGAG | 5′ CTGGTTTAAGCTGCAGAAG |
| | AC 3′ | GC 3′ |
| 4 | 5′ GAAGACTTCGCATCTCCTC | 5′ GCAAGATGAGGCCAACTCA |
| | TG 3′ | A 3′ |
| 5 | 5′ GTACAGCATAACCCGCCAG | 5′ GGATAGTGGTGAGTCACAT |
| | G 3′ | GGA 3′ |
| 6 | 5′ CTTTCTTCTGACCCAGTGA | 5′ CATCTCAGTGTTTGGGTGC |
| | TGG 3′ | CT 3′ |
| 7 | 5′ GTCCAGGAGGGAGAAAAGT | 5′ CTCCTCCTCAGGTTGTGAC |
| | ACC 3′ | C 3′ |
| 8 | 5′CCTGCAGTCTGACCTGAACA | 5′ GGAGGCTCAGAGAGGTCAC |
| | AG 3′ | AT 3′ |
| 9 | 5′ ATCTTGGGAGTGTTTTGCT | 5′ GCCTACTGTCGCAAGGACT |
| | CTG3′ | ACT 3′ |
| 10 | 5′ GCTGCTGAGGTTGGAGTCA | 5′ GAGAGGGGTCTTCCTCATT |
| | G 3′ | GT 3′ |
| 11 | 5′ ATGGGAAACGCCTAGCAAG | 5′ GACTGAAACTGGAACCCTT |
| | TA 3′ | GTC 3′ |

Primers for quantitative real-time PCR

| Gene | Forward | Reverse |
|---|---|---|
| PXN | 5′ TCTTCGAGCGGGATGGA | 5′GCCGGATGAGGAACTCACA |
| | C 3′ | T 3′ |
| MET | 5′ GCTGGTGGTCCTACCATAC | 5′CTGGCTTACAGCTAGTTTG |
| | ATG 3′ | CCA3′ |
| EGFR | 5′GAATTGGGATGCAGAGCTT | 5′GACATGCTGCGGTGTTTT |
| | C 3′ | C3′ |
| LINE-1 | 5′AAAGCCGCTCAACTACATG | 5′ TGCTTTGAATGCGTCCCA |
| | G3′ | GAG 3 |

### Constructs of Wild Type and Mutant Paxillin Plasmids

Paxillin was sub-cloned into pcDNA3.1 DEST47 through Gateway cloning (Invitrogen, Carlsbad, Calif.). Briefly, primers 5′CACCATGGACGACCT CGACGCC 3′ (SEQ ID NO:1) and 5′GCAGAAGAGCTTGAGGAAGCAGTTCTGAC 3′ (SEQ ID NO:2) were used to PCR amplify the paxillin coding region, which was subsequently TOPO cloned into a pENTR/SD/D-TOPO entry vector using LR clonase II. The paxillin insert was transferred into the destination vector, pcDNA3.1 DEST47, by a recombination reaction. The A127T (nucleotide 379 G→A) mutation was independently introduced into paxillin in DEST47 using Quick-Change Site Directed Mutagenesis (Stratagene, LaJolla, Calif.), using the primers (5′CCCCAACAAGCAGAAATCAACTGAGCCTTCACCCACCG 3′ (SEQ ID NO:3), 5′CGGTGGGTGAAGGCTCAGTTGATTTCTGCTTGTTGGGG 3′ (SEQ ID NO:4)). The insert alteration was confirmed by direct sequencing.

### Immunoblotting and Antibodies

Cell lysate preparation, SDS-PAGE, and immunoblot analysis were performed as previously described (Maulik G, Kijima T, Ma P C, et al. Clin Cancer Res 2002;8:620-7.). Polyclonal phosphorylation site-specific paxillin antibodies were obtained from Biosource International (Camarillo, Calif.). Paxillin monoclonal antibody was obtained from Neomarkers, Lab Vision Corp. (Clone 5H11, Fremont, Calif.). Phospho-AKT (S-473) was obtained from Cell Signaling Technology (Beverly, Mass.). c-Met (c-12), Bcl-2 and CD31 antibodies were obtained from Santa Cruz Biotechnology (Santa Cruz, Calif.), and β-actin monoclonal antibody and all other chemicals were purchased from Sigma (St. Louis, Mo.).

### Confocal Microscopy and Immunohistochemistry (IHC)

Cells were grown on glass coverslips, and immunofluorescence procedure performed as previously described (Jagadeeswaran R, Ma P C, Seiwert T Y, et al. Cancer Res 2006;66:352-61.). Images were captured using a confocal microscope and saved as digital images. For immunohistochemistry, paraffin-embedded, formalin-fixed tissue slides and tissue microarrays (TMA) were analyzed using institutional review board approved protocols. Expression of paxillin was quantified manually in each core at ×400 magnification by pathologists and with Automated Cellular Imaging System (ACIS, Clarient, Calif.). Two independent pathologists reviewed all of the staining and immunoscoring was confirmed.

### Fluorescent In Situ Hybridization (FISH)

FISH analysis was done utilizing three different BAC probes: RP11-144B2 is localized to 12q24.23 and contains the full-length paxillin gene; RP11-433C10 is localized to 7p11.2 and contains the full-length EGFR gene; and RP11-163C9 is localized to 7q31.2 and contains the c-Met gene. Two color FISH was done for NSCLC cell lines, utilizing RP11-144B2 (labeled in red) together with RP11-163C9 (labeled in green) and RP11-433C10 together with RP11-163C9. Probes were labeled and fluorescent in situ hybridization was done as previously described (Smolen G A, Sordella R, Muir B, et al. Proc Natl Acad Sci U S A 2006;103:2316-21.). At least 10 metaphase cells were analyzed for each cell line.

### Quantitative Real-Time PCR

Quantitative real-time PCR for gene copy number measurement for DNA samples obtained from tumors of patients with NSCLC was done as described before (Moroni M, Veronese S, Benvenuti S, et al. Lancet Oncol 2005;6:279-86.) using the Stratagene Mx3000P system and the iQ SYBR® green PCR kit (Bio-Rad laboratories, Hercules, Calif.). Relative gene copy number was calculated from the real-time PCR efficiencies, which were determined for each individual run, and the crossing point (CP) deviations of the target and reference genes in a test sample versus a control. LINE-1 (long interspersed element 1) served as reference gene, which is a repetitive element for which copy numbers per diploid genome are similar in healthy or malignant human cells (Wang T L, Maierhofer C, Speicher M R, et al. Proc Natl Acad Sci U S A 2002;99:16156-61.). Primer sets used for PXN, MET, EGFR and LINE-1 are listed in Table 1. Reactions were done in triplicate under standard thermocycling conditions (one cycle of 95° C. for 12 min, followed by 45 cycles of 95° C. for 20 s and 58° C. for 1 min) and the mean threshold cycle number was used.

### Transfection and Serum Starvation Assays

For paxillin transient transfection, H522 cells were plated the day before transfection in six-well plates and transfected, in duplicate, with 2 μg total plasmid DNA and 5 μl of Lipofectamine PLUS (Invitrogen, Carlsbad, Calif.) according to manufacturer's instructions. Cells were serum starved and harvested 24 hours and 48 hours later in lysis buffer. Paxillin levels were assessed by immunoblotting. Cell survival was determined using the MTT assay, as previously described (Ma et al. Cancer Res 2003;63:6272-81.).

For colony survival (clonogenic) studies as described previously (Ma et al. Cancer Res 2003;63:6272-81), transfected H522 cells were reseeded at 0.1×10⁴ cells per well in 6-well plates. After incubation for 4 weeks, the colonies were fixed and stained with methylene blue in 50% ethanol for 20 minutes, then rinsed with water and air-dried. Colonies were counted with a Gel Doc-XR Imager (Bio-Rad laboratories, Hercules, Calif.) using the colony counting program.

### Gene Silencing Assays

SiRNA gene silencing studies were done using methods as previously described (Jagadeeswaran R, Ma P C, Seiwert T Y, et al. Cancer Res 2006;66:352-61.). SiRNA oligonucleotides targeting paxillin mRNA were obtained from Dharmacon, Inc. (Lafayette, Colo.) and used according to the instructions of the manufacturer. Cell viability was measured in triplicates by MTT assay 72 hr after siRNA transfection. Percentage cell viability inhibition by paxillin targeting siRNA was shown with control siRNA in BEAS- 2B and SK-LU-1 cells.

### Xenograft Assays in Nude Mice

We investigated the in vivo oncogenic properties of the mutated paxillin by studying the ability of A127T paxillin and wild type paxillin-stably expressing H522 cells to induce tumor formation in nude mice. Cells expressing wild type paxillin (H522-Wt paxillin), A127T paxillin (H522-A127T paxillin) or control (H522 control vector) were injected subcutaneously into nude mouse flank region (5×10⁶ cells/flank). Animal experiments were done according to institutional approved protocols (IACUC). The animals were monitored for tumor formation every week and sacrificed 8-12 weeks later. Tumor growth was measured every week over a 12-week period.

Tumor morphology, invasion and IHC were studied at the end of the experiment. Tumor tissues were fixed in 10% formalin and embedded in paraffin. Representative tumor sections were obtained from paraffin embedded tumor tissue and stained with H&E, Masson's trichrome and specific antibodies. Areas (n=10) of IHC staining with Ki67, Masson's trichrome and CD31 were analyzed. Expression levels were quantified using the automated cellular imaging system (ACIS, Clarient, Calif.).

### Statistical Analyses

Data are expressed as the mean±S.E.M. Statistical significance was tested using statistics software 15.0 (Chicago, Ill.). For comparison between means of two groups, Student's t test or Chi-Square test was used. For comparing means between more than 2 groups, one-way ANOVA was used. For evaluation of correlations, Spearman's test was used. Unless otherwise stated, representative figures reflect the findings in a minimum of n=3 evaluations and mean values reflect data obtained in a minimum of n=6 mice.

### Results

Level of Paxillin Expression Correlated with Higher Stage and Metastasis

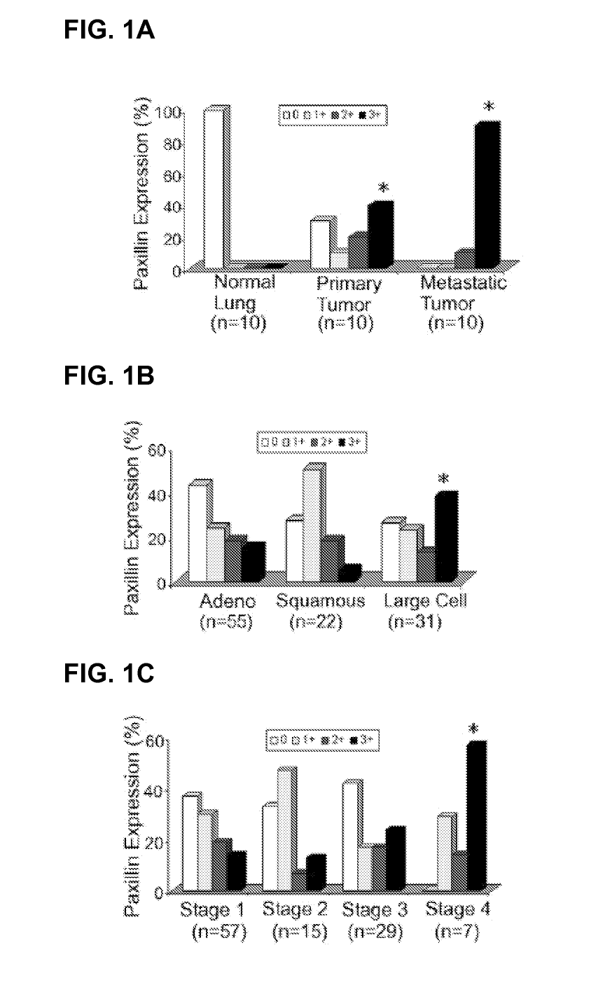

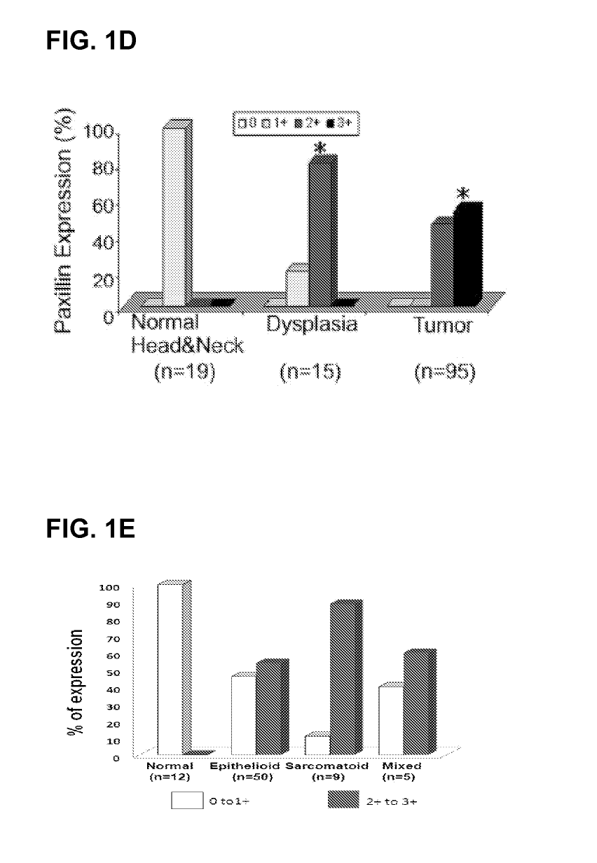

To understand the role of paxillin in lung cancer, paxillin expression was systematically examined using immunohistochemical studies. Paxillin expression was correlated with histology, stage, and metastasis as shown in FIG. 1A. Paxillin intensity measurements were translated into the 4-tier system as negative (score=0), weak (score=1+), moderate (score=2+), or strong (score=3+) staining The y-axis of the graph represents the percentage of cells having the indicated level of paxillin expression. The level of paxillin expression was significantly (P<0.001) higher in lymph node or brain metastatic samples as compared to primary lung tumors. The expression of paxillin in adjacent normal lung of the corresponding tumors was negative. (FIG. 1A). In NSCLC, large cell carcinomas had high expression (score 2+ to 3+) of paxillin (51%) compared to adenocarcinomas (33%) and squamous cell carcinomas (23%) (FIG. 1B). The paxillin expression level increased with increasing stage (FIG. 1C). This did not reach statistical significance (p=0.11) (FIG. 1C). In the samples of primary versus lymph node or brain metastasis, the level of paxillin expression was increased in the metastatic setting. In head and neck squamous cell carcinoma, paxillin levels showed a progressive increase from normal tissue to dysplasia to cancer. (FIG. 1D). In mesothelioma, paxillin levels were increased in epitheliod and mixed tissues as compared to control normal tissues and sarcomatoid tissues expressed the highest levels of paxillin. (FIG. 1E)

### Expression of Paxillin in Cancer Cell Lines

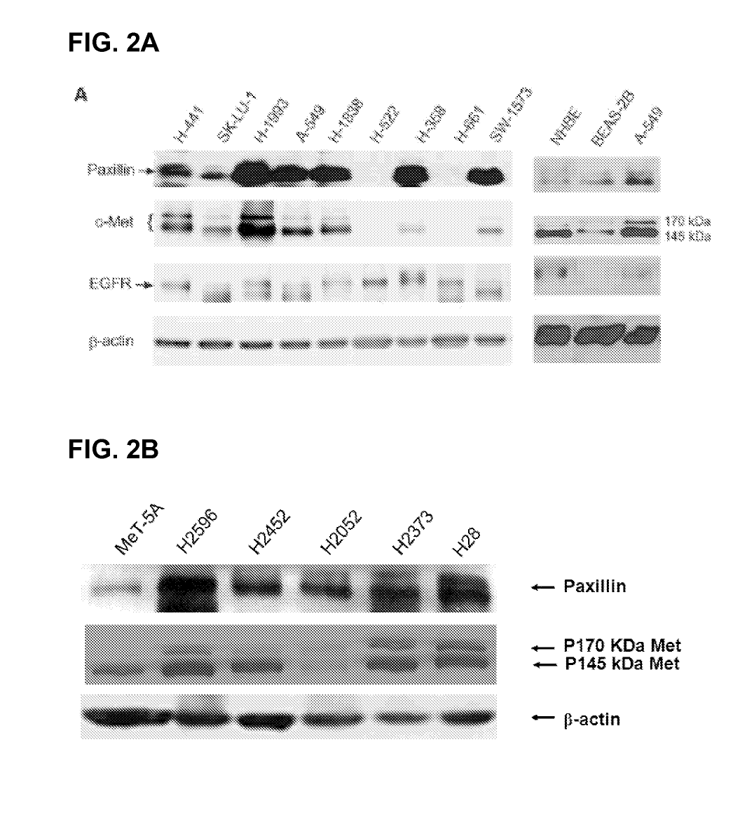

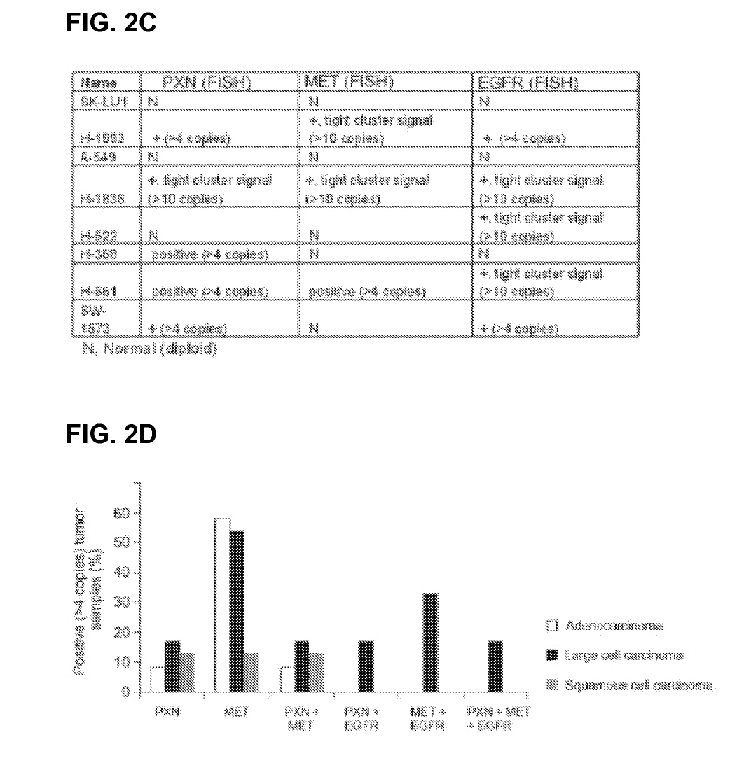

Expression of paxillin, c-Met and EGFR was analyzed by immunoblotting in a panel of lung cancer cell lines and two normal lung epithelial cell lines. There was high expression of paxillin and coexpression of c-Met but not EGFR in NSCLC cell lines. Low level expression of paxillin was observed in NHBE and BEAS-2B cells when compared to A549 (NSCLC) cells (FIG. 2A). The upper 170-kDa protein band of c-Met represents the glycosylated precursor c-Met and the lower 145-kDa band is the biologically active transmembrane β-subunit of c-Met.

Expression of paxillin in mesothelioma cell lines was also analyzed by immunoblotting and compared to a non-malignant cell line (MeT-5A). High level of paxillin and c-Met expression was apparent in each cell line tested. (FIG. 2B)

Gene Copy Numbers of PXN and Correlation with MET and EGFR in Lung Cancer Cell Lines and Tumor Tissues

Gene copy numbers of PXN, MET and EGFR were analyzed by FISH analysis in a panel of lung cancer cell lines. There were increased gene copy numbers of the PXN in H1993, H1838, H358, H661 and SW1573 NSCLC cell lines. The frequency of genomic gain of PXN in lung cancer cell lines was about 62.5% (5 out of 8 cell lines tested). H1993 cells had increased gene copy numbers of paxillin (>4 copies) as well as clustered gene amplification of c-Met (>10 copies). H1838 cells had clustered gene amplification (>10 copies) of PXN and MET (FIG. 2C). There was association between paxillin and c-Met in terms of expression and copy number alterations in most of the NSCLC cell lines tested. We further quantitatively analyzed gene copy numbers of NSCLC tumor tissues from 66 patients for PXN, MET and EGFR using a quantitative real-time P CR method. Histological cell types of these NSCLC tissues included adenocarcinoma (n=26), large-cell carcinoma (n=24), and squamous-cell carcinoma (n=16). 17% of large-cell carcinoma exhibited increased gene copy numbers of PXN and those positive samples also showed increased gene copy numbers of MET (>4 copies) and high copy numbers of EGFR (>10copies). 8% of adenocarcinoma exhibited increased gene copy numbers of PXN and those positive samples showed only high gene copy numbers of MET(>10) but not EGFR. 13% of squamous-cell carcinoma exhibited increased gene copy numbers of PXN and MET. 33% of large-cell carcinoma exhibited increase gene copy numbers of MET and EGFR (FIG. 2D).

In the mesothelioma cell lines tested, three of nine were positive for PXN gene amplification by FISH. Two of the cell lines positive for gene amplification were epitheliod and one was sarcomatoid. Each of these lines had more than 4 copies.

### PXN Mutations in Lung Cancer

To determine whether mutations of the PXN gene were found in lung cancer cells, the paxillin coding region (11 exons) was sequenced in cancer DNA specimens, cancer cell lines and corresponding normal DNA. The paxillin gene was sequenced from DNA samples obtained from corresponding normal tissues identified pathologically as distant normal tissues or adjacent normal tissues through laser capture micro-dissection. A total of 21 paxillin mutations were identified in lung cancer tissue specimens and cell lines (FIG. 3A). The normal tissues showed a wild-type paxillin sequence, confirming that the mutations were somatic in origin.

Some of the mutations were found as homozygous in the SK-LU-1 (A127T paxillin) and H820 (E423K paxillin) cell lines; in addition, one large cell carcinoma tumor tissue (sample T11) contained two homozygous mutations, P233L, and D399N. All other mutations identified were heterozygous.

The overall rate of paxillin mutations in lung cancer was 9.4% (18/191). The frequency of paxillin mutations was high in NSCLC which included large cell carcinomas (18.4% or 7/38), adenocarcinomas (8.6% or 8/93), and squamous cell carcinomas (6% or 3/51). There were no paxillin mutations in the SCLC primary tissue samples examined (0/9). The A127T paxillin mutation, the most frequent mutation detected in NSCLC specimens, was also identified in H69 and H249 SCLC cell lines (2/36 cell lines tested). Overall, the paxillin mutation rate in the 71 lung cancer cell lines tested was 5.6% (4/71) (Table 2).

TABLE 2
Paxillin missense mutations in various tumor
tissue and cell lines

| | Histo- | | Amino | Eth- | |
|---|---|---|---|---|---|
| Tumor | logical | Codon | acid | nic- | Smoking |
| ID | type | change | change | ity | status |
| T3 | large | CCC→CTC, | P50L, | Cc | y |
| | cell ca. | GGA→GAA, | G358E, | | |
| | | CCC→CTC | P487L | | |
| T5 | large | GGC→GAC, | G105D, | AA | y |
| | cell ca. | GTG→ATG | V395M | | |
| T6 | large | GGG→GAG, | G485E, | AA | y |
| | cell ca. | AAG→AGG | K506R | | |
| T11 | large | CCT→CTT, | P233L, | AA | y |
| | cell ca. | GAC→AAC | D399N | | |
| T13 | large | CCC→TCC | P30S | Cc | y |
| | cell ca. | | | | |
| T14 | large | CCC→CTC | P52L | Cc | y |
| | cell ca. | | | | |
| T27 | large | CCC→CTC | P47L | AA | y |
| | cell ca. | | | | |
| T39 | squ. cell | CCA→TCA | P46S | AA | y |
| | ca. | | | | |
| T42 | squ. cell | GAG→AAG, | E355K, | Cc | y |
| | ca. | GAG→AAG | E356K | | |
| T52 | squ. cell | ACA→ATA | T255I | Cc | y |
| | ca. | | | | |
| T103 | adeno- | GGC→AGC | G388S | AA | y |
| | carcinoma | | | | |
| T105 | adeno- | TTC→TCC | F408S | Cc | y |
| | carcinoma | | | | |
| T112 | adeno- | CCC→TCC, | P30S, | Cc | y |
| | carcinoma | CCC→CTC, | P52L, | | |
| | | GCT→ACT | A127T | | |
| T136 | adeno- | GCT→ACT | A127T | Cc | y |
| | carcinoma | | | | |
| T146 | adeno- | TCA→TTA | S32L | Cc | y |
| | carcinoma | | | | |
| T171 | adeno- | GCT→ACT | A127T | Cc | — |
| | carcinoma | | | | |
| T192 | H & N | CCC→CTC | P52L | — | — |
| | cancer | | | | |
| T194 | Mesothe- | GGG→AGG | G297R | Cc | n |
| | lioma | | | | |
| T243 | Mesothe- | GCT→ACT | A127T | Cc | y |
| | lioma | | | | |
| SK- | adeno- | GCT→ACT | A127T | Cc | — |
| LU-1 | carcinoma | | | | |
| H 820 | adeno- | GAG→AAG | E423K | Cc | — |
| | carcinoma | | | | |
| H69 | SCLC | GCT→ACT | A127T | Cc | — |
| H249 | SCLC | GCT→ACT | A127T | Cc | — |

Note:
Cc, Caucasian; AA, African American; y, Smoker; n, non smoker; —, Not known; Median Age: 64 years (range 48-77 years) Male/Female (53%: 47%).

PXN Mutations in Malignancies Other than Lung Cancer

Paxillin mutations were not as frequent in many other cancers, including mesothelioma (rate of 2/73), head and neck cancer (1/10 at G139S), breast cancer (0/16), melanoma (0/4), cervical cancer (0/6), myelodysplastic syndrome (0/20), and chronic myelogenous leukemia (0/22). The overall mutational rate for these malignancies, not including lung cancer, was 2% (3/151).

### Ethnic Variations and Nature of PXN Mutations

Most of the identified paxillin mutations (19/21) clustered in the region between LD motifs 1 and 2 (amino acid residues Pro30 to Gly139) and the region spanning the LIM domains (FIG. 3A). In lung cancer tissues examined, the paxillin mutations were identified in samples from Caucasians and African-Americans, each with a unique mutational spectrum. However, there were no mutations for paxillin identified in Taiwanese samples (0/70). FIG. 3B shows the differences between ethnic groups in paxillin mutations. The mutational spectrum of paxillin was characterized by a high proportion of GC to AT (97%) transitions (FIG. 3C). We have observed 3% of AT to GC, 97% of GC to AT transition, and 0% AT to TA or GC to CG changes.

The paxillin mutations identified as shown in Table 2 have not been reported previously and are not present in the NCBI-SNP database. In addition, synonymous SNPs were identified which are distinct from previously known synonymous SNPs, and non-synonymous SNPs such as S73G, were also identified for paxillin and differences were noticed in the allele frequency in various ethnic groups (Table 3).

TABLE 3
Frequencies of paxillin SNPs in ethnic groups

| | T39T | P50P | | N238N | F276F | R382R^{#} | V394V | T509T^{#} |
|---|---|---|---|---|---|---|---|---|
| Ethnicity | 191 | 224 | S73G* | 788 | 902 | 1220 | 1256 | 1601 |
| Caucasian | 0 | 0.7% | 96.4% | 0.7% | 0.7% | 0 | 0.7% | 5.8% |
| (n = 300) | | | | | | | | |
| African | 2.4% | 0 | 100% | 0 | 0 | 2.4% | 0 | 0 |
| American | | | | | | | | |
| (n = 42) | | | | | | | | |
| Asian | 0 | 0 | 100% | 0 | 0 | 0 | 0 | 0 |
| (Taiwanese) | | | | | | | | |
| (n = 70) | | | | | | | | |

*Previously reported non synonymous paxillin SNP
^{#}Previously reported synonymous paxillin SNP

### A127T Paxillin Mutant Function In Vitro in Lung Cancer

### Increased Cell Growth and Colony Formation

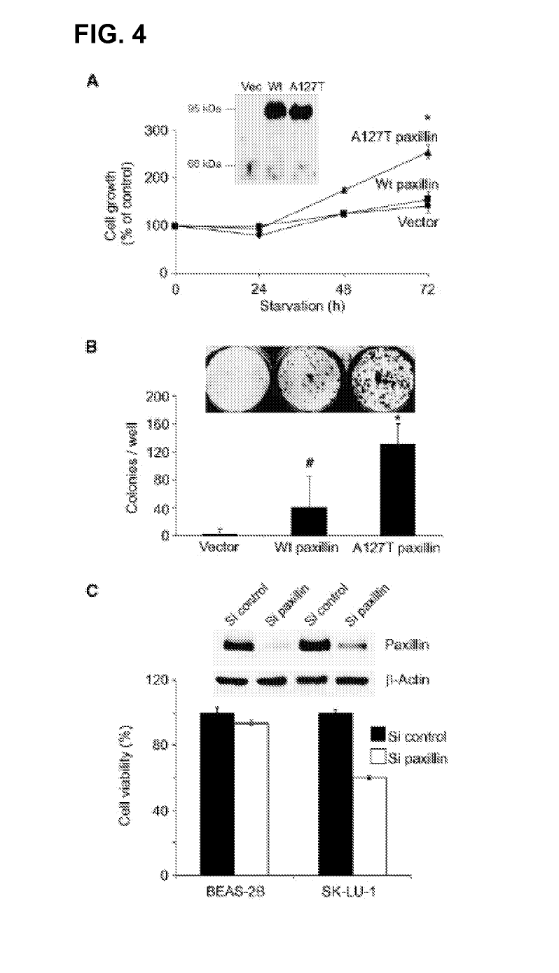

To investigate the role of paxillin and the A127T mutant form of paxillin in lung cancer, the H522 paxillin negative cell line was stably transfected to express wild-type and mutant EGFP-paxillin fusions. Presence of the higher molecular weight (95 kDa) EGFP fusion paxillin was confirmed by immunoblotting. The viability of H522 cells expressing wild type paxillin was comparable to that of control vector cells when subjected to starvation. H522 cells expressing A127T paxillin grew more rapidly (two-fold faster) than cells expressing wild type paxillin or the control vector (FIG. 4A). This was corroborated by the significantly higher number of colonies formed by H522 cells expressing A127T paxillin as compared to cells expressing wild type paxillin or control vector (FIG. 4B). These data demonstrate that cells expressing the A127T paxillin have increased cell growth and colony formation as compared to wild-type cells expressing wild-type paxillin.

Decreased Cell Viability with PXN siRNA

Paxillin knock-down was effectively achieved in both the BEAS-2B (normal bronchial epithelial cells) and SK-LU-1 cell lines by 70% to 90% after 72 hours of paxillin specific-siRNA transfection compared to control siRNA transfection. A significant reduction (40%±1.3%) of cell viability was observed in paxillin siRNA- transfected SK-LU-1 cells compared to BEAS-2B cells (6.5%±1.5%) (FIG. 4C). These data suggest that increased expression of paxillin is necessary for the increased cell viability.

Redistribution of Mutant PXN and Association with Bcl-2

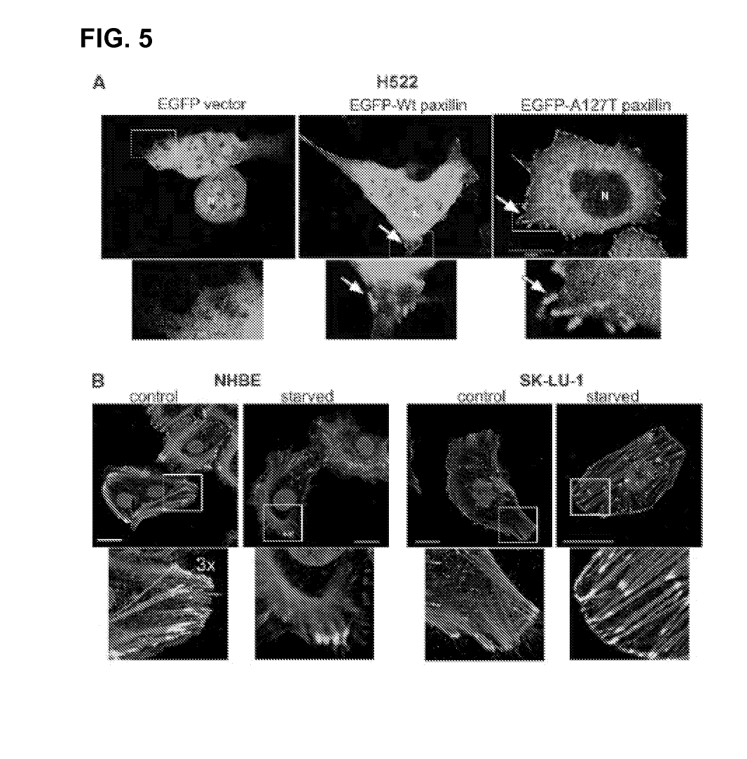

Paxillin localization was examined using confocal microscopy. H522 cell line was chosen as representative of NSCLC, and exhibited minimal to no paxillin expression. In H522 cells, localization of exogenously expressed wild-type paxillin was diffuse throughout the intracellular compartments in the cell. In contrast, A127T paxillin expression was localized toward the cell periphery and had the characteristic punctate appearance of focal adhesion points (FIG. 5A). We further studied paxillin localization using non-immortalized primary normal human bronchial epithelial (NHBE) cells and compared with localization of A127T paxillin mutant SK-LU-1 cells. A well-defined actin cytoskeleton was observed in control NHBE cells and was found to be disrupted with reduced paxillin focal adhesion and F-actin fibers after starvation. In contrast, massive stress fiber formation with redistribution of paxillin to the ends of stress fibers occurred in SK-LU-1 cells (harboring the A127T paxillin mutant). Redistribution of paxillin and enhancement of the cortical actin ring were noticed predominantly in starved SK-LU-1 cells (FIG. 5B). Since mutant paxillin staining in the cytoplasm resembled the staining seen with mitochondria, we determined whether paxillin localized to the mitochondria and colocalized with Bcl-2 using Mitotracker red dye and antibody specific to Bcl-2, respectively. In SK-LU-1 cells, A127T mutant co-localized with Bcl-2 in the mitochondria after starvation. In NHBE cells, co-localization of paxillin and Bcl-2 in the mitochondria was not observed.

### Paxillin or A127T Paxillin Increases Tumor Growth and Invasion In Vivo

In vivo properties of A127T paxillin and wild type paxillin-expressing cells in nude mice were determined. H522 cells expressing wild-type paxillin or A127T paxillin and control vector group of xenografts were generated. Tumor growth was measured every week up to 12 weeks. At 12 weeks, tumor growth in the A127T mutant paxillin group markedly exceeded (P=0.0019) that in the control vector group or wild-type paxillin (FIG. 6A). Paxillin negative H522 cells grew in nude mice as a solid mass without any invasion, whereas either wild-type paxillin or A127T paxillin mutant expressing H522 cells grew as nodular tumors. In addition to nodularity, A127T paxillin expressing H522 xenograft tumors were highly invasive into the adjacent muscle tissue. Upon gross examination, the A127T paxillin tumors had larger nodules as compared to the wild-type paxillin tumors. Immunohistochemical analysis (IHC) of tumor sections from mice in various groups were examined using an antibodies specific for nuclear antigen Ki-67 (a marker for active cell division) and CD31 (measuring microvessel density). IHC of xenografts revealed that as compared to control vector H522, there was enhanced cell proliferation, increased stroma, and increased microvessel density in wild type paxillin H522 xenografts. In the A127T paxillin H522 xenografts, as compared to paxillin negative or paxillin positive cells, there was enhanced cell proliferation with less stroma and microvessel density (FIG. 6B).

1. A method for assessing risk of metastasis of a tumor in a mammal, comprising determining the paxillin gene copy number or detecting the presence of a paxillin mutation in the tumor, the presence of an increased number of paxillin gene copies or a paxillin mutation being indicative of an increased risk of metastasis.

2. The method of claim 1, wherein the paxillin gene copy number is determined by FISH.

3. The method of claim 1, wherein the paxillin gene copy number is determined by real time PCR.

4. The method of claim 1, wherein the tumor is a lung cancer tumor.

5. The method of claim 4, wherein the lung cancer tumor is a large cell carcinoma.

6. (canceled)

7. The method of claim 1, wherein the tumor is a lung cancer tumor, a head and neck cancer tumor, or a mesothelioma tumor and the presence of a paxillin mutation is detected.

8. The method of claim 7, wherein the lung tumor is a small cell lung cancer tumor or a non-small cell lung cancer tumor.

9. The method of claim 8, wherein the non-small cell lung cancer tumor is a large cell carcinoma, an adenocarcinoma, or a squamous cell carcinoma.

10. The method of claim 1, wherein the paxillin mutation is located between two LD motifs, or in a LIM domain of the paxillin.

11. (canceled)

12. The method of claim 10, wherein the paxillin mutation comprises a mutation selected from the group consisting of P30S, S32L, P46S, P46L, P47L, P47S, P50L, P52L, G105D, A127T, G139S, P233L, T255I, G297R, E355K, E356K, G358E, G388S, V395M, D399N, F408S, E423K, P487L, and K506R.

13. The method of claim 10, wherein the paxillin mutation comprises A127T.

14. The method of claim 10, wherein the paxillin mutation is a single nucleotide polymorphism (SNP).

15. The method of claim 14, wherein the SNP does not alter the amino acid sequence of paxillin.

16. The method of claim 14, wherein the SNP is a non-coding nucleotide change at a nucleotide of SEQ ID NO:36 selected from the group consisting of nucleotide position numbers 191, 224, 788, 902, 1220, 1256 and 1601.

17. The method of claim 1, wherein the paxillin mutation is a GC to AT transition.

18. A method of staging a tumor in a mammal, comprising: (a) detecting the presence of a paxillin mutation in the tumor; and (b) correlating the presence of the paxillin mutation in the tumor with the stage of the tumor.

19. The method of claim 18, wherein the tumor is selected from the group consisting of a small cell lung cancer tumor, a large cell carcinoma, an adenocarcinoma, a squamous cell carcinoma, a head and neck cancer tumor, or a mesothelioma tumor.

20. (canceled)

21. (canceled)

22. The method of claim 18, wherein the paxillin mutation is located between two LD motifs, or in a LIM domain of the paxillin.

23. The method of claim 18, wherein the paxillin mutation is selected from the group consisting of P30S, S32L, P46S, P46L, P47L, P47S, P50L, P52L, G105D, A127T, G139S, P233L, T255I, G297R, E355K, E356K, G358E, G388S, V395M, D399N, F408S, E423K, P487L, and K506R.

24. The method of claim 18, wherein the paxillin mutation is a single nucleotide polymorphism (SNP), the SNP is selected from the group consisting of nucleotide position numbers 191, 224, 788, 902, 1220, 1256 and 1601.

25. (canceled)

26. An isolated oligonucleotide at least 16 bases in length and comprising a paxillin mutation at a nucleotide of SEQ ID NO:36 selected from the group consisting of 162, 169, 191, 210, 211, 213, 214, 223, 224, 229, 388, 453, 489, 772, 788, 838, 902, 963, 1137, 1140, 1147, 1220, 1236, 1256, 1257, 1269, 1297, 1341, 1528, 1534, 1591, and 1601.

27-32. (canceled)


SEQUENCE LISTING (37 sequences)

SEQ ID NO: 1 - DNA, 22 bp, Artificial
  Oligonucleotide
caccatggacgacctcgacgcc

SEQ ID NO: 2 - DNA, 29 bp, Artificial
  Oligonucleotide
gcagaagagcttgaggaagcagttctgac

SEQ ID NO: 3 - DNA, 38 bp, Artificial
  Oligonucleotide
ccccaacaagcagaaatcaactgagccttcacccaccg

SEQ ID NO: 4 - DNA, 38 bp, Artificial
  Oligonucleotide
cggtgggtgaaggctcagttgatttctgcttgttgggg

SEQ ID NO: 5 - DNA, 20 bp, Artificial
  Oligonucleotide
cagctagcgcgaccctgagc

SEQ ID NO: 6 - DNA, 24 bp, Artificial
  Oligonucleotide
gaatggtcctgcaccagtaatatg

SEQ ID NO: 7 - DNA, 24 bp, Artificial
  Oligonucleotide
ggtgctagaaggcattaactcttg

SEQ ID NO: 8 - DNA, 20 bp, Artificial
  Oligonucleotide
cttagctggcaggggcttag

SEQ ID NO: 9 - DNA, 21 bp, Artificial
  Oligonucleotide
gtggaaattccctggtgagac

SEQ ID NO: 10 - DNA, 21 bp, Artificial
  Oligonucleotide
ctggtttaagctgcagaaggc

SEQ ID NO: 11 - DNA, 21 bp, Artificial
  Oligonucleotide
gaagacttcgcatctcctctg

SEQ ID NO: 12 - DNA, 20 bp, Artificial
  Oligonucleotide
gcaagatgaggccaactcaa

SEQ ID NO: 13 - DNA, 20 bp, Artificial
  Oligonucleotide
gtacagcataacccgccagg

SEQ ID NO: 14 - DNA, 22 bp, Artificial
  Oligonucleotide
ggatagtggtgagtcacatgga

SEQ ID NO: 15 - DNA, 22 bp, Artificial
  Oligonucleotide
ctttcttctgacccagtgatgg

SEQ ID NO: 16 - DNA, 21 bp, Artificial
  Oligonucleotide
catctcagtgtttgggtgcct

SEQ ID NO: 17 - DNA, 22 bp, Artificial
  Oligonucleotide
gtccaggagggagaaaagtacc

SEQ ID NO: 18 - DNA, 20 bp, Artificial
  Oligonucleotide
ctcctcctcaggttgtgacc

SEQ ID NO: 19 - DNA, 22 bp, Artificial
  Oligonucleotide
cctgcagtctgacctgaacaag

SEQ ID NO: 20 - DNA, 21 bp, Artificial
  Oligonucleotide
ggaggctcagagaggtcacat

SEQ ID NO: 21 - DNA, 22 bp, Artificial
  Oligonucleotide
atcttgggagtgttttgctctg

SEQ ID NO: 22 - DNA, 22 bp, Artificial
  Oligonucleotide
gcctactgtcgcaaggactact

SEQ ID NO: 23 - DNA, 20 bp, Artificial
  Oligonucleotide
gctgctgaggttggagtcag

SEQ ID NO: 24 - DNA, 21 bp, Artificial
  Oligonucleotide
gagaggggtcttcctcattgt

SEQ ID NO: 25 - DNA, 21 bp, Artificial
  Oligonucleotide
atgggaaacgcctagcaagta

SEQ ID NO: 26 - DNA, 22 bp, Artificial
  Oligonucleotide
gactgaaactggaacccttgtc

SEQ ID NO: 27 - DNA, 18 bp, Artificial
  Oligonucleotide
tcttcgagcgggatggac

SEQ ID NO: 28 - DNA, 20 bp, Artificial
  Oligonucleotide
gccggatgaggaactcacat

SEQ ID NO: 29 - DNA, 22 bp, Artificial
  Oligonucleotide
gctggtggtcctaccatacatg

SEQ ID NO: 30 - DNA, 22 bp, Artificial
  Oligonucleotide
ctggcttacagctagtttgcca

SEQ ID NO: 31 - DNA, 20 bp, Artificial
  Oligonucleotide
gaattgggatgcagagcttc

SEQ ID NO: 32 - DNA, 19 bp, Artificial
  Oligonucleotide
gacatgctgcggtgttttc

SEQ ID NO: 33 - DNA, 20 bp, Artificial
  Oligonucleotide
aaagccgctcaactacatgg

SEQ ID NO: 34 - DNA, 21 bp, Artificial
  Oligonucleotide
tgctttgaatgcgtcccagag

SEQ ID NO: 35 - protein, 557 aa, Homo sapiens
MDDLDALLADLESTTSHISKRPVFLSEETPYSYPTGNHTYQEIAVPPPVPPPPSSEALNGTILDPLDQWQPSGSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 36 - DNA, 3595 bp, Homo sapiens
  CDS (75)..(1748): PXN
aaaagttgcggggcatagacgagcgccccgggacgcagctagcgcgaccctgagccggcgcccgtggtccggccatggacgacctcgacgccctgctggcggacttggagtctaccacctcccacatctccaaacggcctgtgttcttgtcggaggagaccccctactcatacccaactggaaaccacacataccaggagattgccgtgccaccccccgtccccccacccccgtccagcgaggccctcaatggcacaatccttgaccccttagaccagtggcagcccagcggctcccgattcatccaccagcagcctcagtcctcatcacctgtgtacggctccagtgccaaaacttccagtgtctccaaccctcaggacagtgttggctctccgtgctcccgagtgggtgaggaggagcacgtctacagcttccccaacaagcagaaatcagctgagccttcacccaccgtaatgagcacgtccctgggcagcaacctttctgaactcgaccgcctgctgctggaactgaacgctgtacagcataacccgccaggcttccctgcagatgaggccaactcaagccccccgcttcctggggccctgagccccctctatggtgtcccagagactaacagccccttgggaggcaaagctgggcccctgacgaaagagaagcctaagcggaatgggggccggggcctggaggacgtgcggcccagtgtggagagtctcttggatgaactggagagctccgtgcccagccccgtccctgccatcactgtgaaccagggcgagatgagcagcccgcagcgcgtcacctccacccaacagcagacacgcatctcggcctcctctgccaccagggagctggacgagctgatggcttcgctgtcggatttcaagttcatggcccaggggaagacagggagcagctcaccccctggggggcccccgaagcccgggagccagctggacagcatgctggggagcctgcagtctgacctgaacaagctgggggtcgccacagtcgccaaaggagtctgcggggcctgcaagaagcccatcgccgggcaggttgtgaccgccatggggaagacgtggcaccccgagcacttcgtctgcacccactgccaggaggagatcggatcccggaacttcttcgagcgggatggacagccctactgtgaaaaggactaccacaacctcttctccccgcgctgctactactgcaacggccccatcctggataaagtggtgacagcccttgaccggacgtggcaccctgaacacttcttctgtgcacagtgtggagccttctttggtcccgaagggttccacgagaaggacggcaaggcctactgtcgcaaggactacttcgacatgttcgcacccaagtgtggcggctgcgcccgggccatcctggagaactatatctcagccctcaacacgctgtggcatcctgagtgctttgtgtgccgggaatgcttcacgccattcgtgaacggcagcttcttcgagcacgacgggcagccctactgtgaggtgcactaccacgagcggcgcggctcgctgtgttctggctgccagaagcccatcaccggccgctgcatcaccgccatggccaagaagttccaccccgagcacttcgtctgtgccttctgcctcaagcagctcaacaagggcaccttcaaggagcagaacgacaagccttactgtcagaactgcttcctcaagctcttctgctaggtgccctgcccctgtctctgccccccttccccagccagcatccccaactgcgactgtgacctagagacttcacccgggggtgaaggggtaaacccgactgaaactggaacccttgtcctccgctggtgcgggatggacagagggccgtgaggggtccccctgcttgtcttcacccctgccagagcctctgggccccctcctccctcctgtagctctccctaggctgcccactctccatcctccccaggggtagaggctgggggctccaccccagcccatgtacgtccccacgaactggcctggccagcaccccacactggagccatctcttcctcatatttcagcagtgcagccggggggcagggaagggcaggcagggtctgttggggtctctttttatccttattcctcccccgacctaattgtctttgttctgtgattattgggggacacccggctccctccagacaatgccagcataaatccatccatccaaaggcagagaaccaaaggggccatggaaggttctctgtgctcctcctacccttccagtgccctaggcctggcgactgcccctgccttttagacccgcactccccttttatacctgctcttgttctactgagaaaagcctctccagcaataatgttttctagtcacttcctccgtctccgggacggcgtgcctggacactgtccgactttgatagatttctacactgaggttgaattcatatcgcctgagatgttttacttctctatacaccatgattttgtagagatattaaagacgttcccttttgtatctcttcttcatcaccgccactgggccttcactgatggtgtctggtgtgtagattgctttgtctgtggggtggggtggggaagcaatatgtattttattgtttcttagcacaagcaggtgtgctgggagcagctctgtgactccccctctttcacttcatagctcccaggactgttttataaactgctgctatttggaaaccccttctttacttcccaggccagcaagccttcactgaaactggttgaagagtgttgcacccttttgggcctagaattctgaactttatctgttctgtttctgtgggaggagaaggggaagtatgttttggggggctgcttcctgtctgagtaagccctcaggagcctcttgctcccctgtgaacccctgaaccttctgagcccccctgcttctatggggctctctcttctgccttctcaggaaagctgtgtctgattttggccatcaggactctgacgtctctttggtcttgttgatttacctctgggcatatcccttccccagatctgctcctcccctttcacaggtgggatcggcactcagggggtctggaaagaaggtcataagggagcatgataggatttggggcagagggacaggctcctctggggaaaccccccagagctctttaccaaggatgaaagaggagccaggccttgggctcctgatgaccagaaaggggccaccggggtctaatggtgacagtccaaaccactccactggcctcctggcagaagccgagtgtgctggggtctccgaagagggtccctcctttttgggggaaggtcagcccagcccctccaaaggtctgatgtctccactttcacccgcaggccttaccgctctgtttatagtgacccaccctagatcttccccaagagggactggggtttctggggtccattctctgagtcagtggttatttgaaaatttgattttgattttattttttctctgtaaacttccaagctggcttttcccatttcaattcctgtgatttatgccaataaagtttgcccatg

SEQ ID NO: 37 - protein, 557 aa, Homo sapiens; identical to SEQ ID NO: 35